(19) United States
(12) Patent Application Publication
(10) Pub. No.: US 2009/0112152 A1
(43) Pub. Date: Apr. 30, 2009

(54) NEEDLELESS ACCESS PORT VALVE

(76) Inventor: Peter W. Peppel, Nazareth, PA (US)

Correspondence Address: KLEIN, O'NEILL & SINGH, LLP, 43 CORPORATE PARK, SUITE 204, IRVINE, CA, 92606, US

(21) Appl. No.: 11/924,921

(22) Filed: Oct. 26, 2007

(51) Int. Cl. A61M 39/02

(52) U.S. Cl. 604/33

(57) ABSTRACT

Needleless access port valves are generally discussed herein with particular discussions extended to needleless access port valves having a resilient piston. The valve includes a valve housing having an inlet port, an outlet port, and an interior cavity. A resilient piston made of a homogeneous material is slidably mounted on a pin and positioned within the interior cavity. The piston is compressed between the pin and the housing.

Needleless access port valves are generally discussed herein with particular discussions extended to needleless access port valves comprising a resilient piston.

### BACKGROUND

Needleless access port valves are widely used in the medical industry for accessing an intravenous (IV) line and/or the internals of a patient or subject. Generally speaking, prior art valves utilize a valve housing in combination with a moveable internal plug or piston to control the flow of fluid through the valve. The plug or piston may be moved by a syringe or a medical implement to open the inlet of the valve for accessing the interior cavity of the valve. When a fluid is delivered through the valve, fluid flow typically flows around the outside of the plug or piston in the direction towards the outlet. Upon removal of the syringe or medical implement, the plug or piston returns to its original position, either un-aided or aided by a biasing means, such as a spring or a diaphragm.

In some prior art valves, when the syringe or medical implement pushes the plug or piston, the plug or piston is pierced by a piercing device, such as a spike. The spike typically incorporates one or more fluid channels for fluid flow flowing through the pierced piston and then through the fluid channels in the spike. In yet other prior art valves, a self-flushing or positive flush feature is incorporated to push residual fluids confined inside the interior cavity of the valve to flow out the outlet when the syringe or medical implement is removed.

While prior art needleless access port valves are viable options for their intended applications, there remains a need for alternative needleless access port valves.

### SUMMARY

The present invention may be implemented by providing a needleless access port valve comprising a valve housing comprising an inlet port adapted to receive a medical implement, an outlet port, and an interior wall surface defining an interior cavity; a resilient piston having a generally tubular shape defining an interior space, wherein the piston comprises a neck section, a middle section, and a lower section; and a pin occupying, at least in part, the interior space of the piston. The lower section of the piston is compressed between the pin and the housing, and the neck section of the piston forms a seal with the housing to prevent fluid flow between the inlet port and the outlet port when in a closed position.

In another embodiment of the present invention, a needleless access port valve comprises a valve housing having an inlet port, an outlet port, an interior cavity, and at least one flow channel; a pin; and a resilient piston formed over the pin and positioned in the interior cavity of the housing. The piston comprises an upper neck section, a mid-section, and a lower section, and the upper neck section comprises an opening configured to align with the flow channel of the valve housing when the valve is in an open position. The lower section is compressed between the pin and the housing to hold the piston in place, and the mid-section is configured to expand outwardly away from the pin when the piston is compressed to open a fluid pathway between the inlet port and the outlet port.

In yet another embodiment of the present invention, a needleless access port valve comprises a valve housing comprising an inlet port, an outlet port, an upper neck section, and an interior cavity, wherein the upper neck section comprises at least one flow channel; a pin; and a resilient piston slidably mounted on the pin and positioned in the interior cavity of the valve housing, wherein the piston comprises an upper sliding section, a middle expanding section, and a lower mounting section. The lower mounting section is compressed between the pin and the housing, and the middle expanding section is adapted to expand away from the pin when the piston is compressed. The upper sliding section is configured to slide along the pin when the piston is compressed, and the upper sliding section of the piston comprises a side opening, which is configured to align with the flow channel when the piston is compressed to open a fluid pathway between the inlet port and the outlet port.

In still another embodiment of the present invention, a needleless access port valve comprises a housing having a top opening, a bottom opening, a lower neck section, and a central cavity, and a resilient piston wedged or fixed in place inside the housing at the lower neck section. When compressed by a medical implement, the piston expands outwardly into the central cavity of the housing, opening a fluid flow channel between the top opening and the bottom opening.

In another embodiment of the present invention, a needleless access port valve comprises a valve housing made of a rigid plastic material and a resilient piston made of a resilient and pliable material capable of substantially recovering its size and shape when deflected or compressed, such as a thermoplastic elastomer.

In another embodiment of the present invention, a needleless access port valve comprises a valve housing having an upper flow channel, a top opening, a bottom opening, and a central cavity. The valve also comprises a pin having upper and lower flow channels and a piston having a side hole in a top section of the piston. The side hole in the piston aligns with the upper flow channel of the valve housing when the piston is compressed. This alignment opens a flow channel from the top opening of the valve housing, through the upper flow channel of the valve housing, through the side hole of the piston, through the upper flow channel of the pin, through the central cavity of the valve housing, through the lower flow channel of the pin, and out the bottom opening of the valve housing.

Other features and variations of the valve assemblies summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is a cross-sectional side view of a needleless access port valve in an embodiment of the present invention, shown in a closed position;

FIG. 2 is a perspective view of the valve of FIG. 1;

FIG. 3 is a cross-sectional side view of the valve of FIG. 1 in an open position; and

FIG. 4 is a perspective view of the valve of FIG. 3.

### DETAILED DESCRIPTION

The detailed description set forth below in connection with the appended drawings is intended as a description of the presently preferred embodiments of needleless access port valves or backcheck valves (herein “valves”) provided in accordance with the present invention and is not intended to represent the only forms in which the present invention may be constructed or utilized. The description sets forth the features and the steps for constructing and using the valves of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like elements or features.

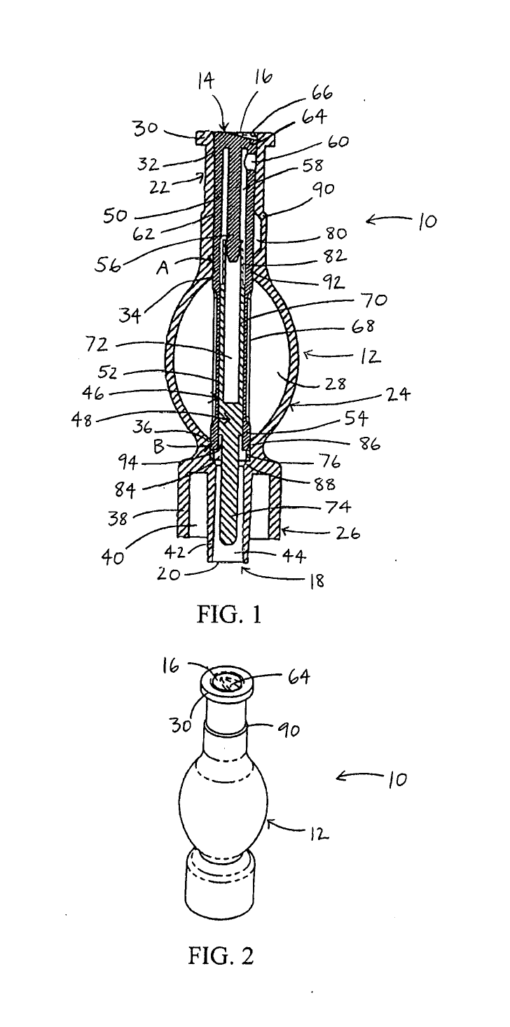

FIG. 1 shows a cross-sectional side view of a needleless access port valve 10 in an embodiment of the present invention. The valve 10 is shown in a closed position. The valve 10 includes a valve housing 12 which may be formed from a rigid plastic material, such as polycarbonate, polyurethane, or the like. In one exemplary embodiment, one or more colors are incorporated into the material. Preferably, the material has a translucent pantone green tone. Alternatively, an opaque material with one or more color tones may also be incorporated. In the embodiment shown in FIG. 1, the housing 12 is integrally formed in one piece. In other embodiments, the housing 12 could be formed from two or more separate sections that are attached to one another in a friction fit, a threaded fit, or other methods known to those skilled in the art, such as gluing or laser or ultrasonic welding.

The valve housing 12 comprises an inlet port 14 having an inlet opening 16 and an outlet port 18 having an outlet opening 20. Fluid is introduced at the inlet port 14 via a medical implement, such as a syringe, and the fluid flows through the valve 10 and exits the outlet port 18. Alternatively, a fluid or blood sample can be withdrawn through the valve 10, such that fluid flows in the direction from the outlet port 18 to the inlet port 14.

In one exemplary embodiment, the valve housing 12 further comprises an upper neck section 22, a bulbous midsection 24, and a lower port section 26. The upper neck section 22 has an upper interior wall 32 which includes an upper flow channel 80 on one side. The upper flow channel 80 may be formed by molding an indentation into the upper interior wall 32. The upper neck section 22 may also comprise exterior luer threads 30 for threaded engagement with a corresponding male threaded luer, and a circular step 90 that acts as a stop for limiting thread engagement with the corresponding male threaded luer.

The bulbous midsection 24 of the housing 12 defines an interior cavity 28. An upper annular shoulder 34 is formed at the upper portion of the bulbous midsection 24, and a corresponding lower annular shoulder 36 is formed at the lower portion of the bulbous midsection 24. The bulbous midsection 24 has an interior diameter that is larger than an interior diameter of the upper neck section 22.

The lower port section 26 of the valve housing 12 comprises an outlet nozzle 42 and an outlet passage 44 through which the fluid flows toward or from the outlet opening 20. The lower port section 26 further comprises a collar 38 defining an annular space 40. The collar 38 and annular space 40 are adapted to receive a female luer from an IV set, a tubing, a catheter assembly, or the like (not shown), which carries fluid to or from the patient. Although not shown, the collar 38 may incorporate internal threads for threaded engagement with a threaded female luer. The lower port section 26 also comprises an interior annular step 88 at the upper end of the outlet passage 44.

A piston 46 and a centering pin 48 are positioned in the interior cavity 28 of the valve housing 12. The piston 46 has a generally tubular shape, forming a generally hollow interior for mounting the piston onto the centering pin. In one exemplary embodiment, the piston 46 is formed of a resilient and pliable material capable of substantially recovering its size and shape when deflected or compressed. In a preferred embodiment, the piston 46 is integrally formed from a thermoplastic elastomer (TPE) such as the copolyamide (COPA) family of thermoplastic elastomers. In a preferred embodiment, the COPA is copolyamide thermoplastic elastomer having a commercial trade name PEBAX®. However, other TPEs may also be used to make the piston 46, including thermoplastic polyurethanes (TPUs), styrenic thermoplastic elastomers, thermoplastic polyolefins (TPOs), copolyesters (COPEs), and thermoplastic vulcanizate elastomeric alloys (TPVs). Optionally, the TPEs may be cross-linked either chemically or by irradiation to alter their characteristics. More particularly, the piston 46 is formed from a silicone elastomer.

The piston 46 comprises an upper plug section 50, a middle bladder section 52, and a lower ring section 54. The upper plug section 50 has a hollow interior and a stem 56 disposed therein, which is preferably integrally formed. The stem 56 forms an annular channel 58 between the stem 56 and the plug section 50. The plug section 50 also comprises a circumferential exterior wall surface 62 and a tapered top surface 64. The tapered top surface 64 provides a fluid flow space between the top surface of the piston 46 and a circumferential end of a medical implement. A gap 66 is formed at the inlet opening 16 due to the relative geometries between the tapered surface 64 and the inlet opening 16. When the valve 10 is in the closed position, shown in FIG. 1, the top of the tapered surface 64 of the piston 46, more particularly the generally planar surface of the top surface, is preferably flushed with the inlet opening 16, except for the gap 66. This configuration allows the top 64 to be swabbed or sanitized. The plug section 50 also includes a side opening 60 which forms a pathway into the annular channel 58.

The middle bladder section 52 comprises a groove 68 formed in the outer surface of the bladder section 52. In exemplary embodiments of the invention, a plurality of grooves 68 are provided. In the particular embodiment shown in FIG. 1, the bladder section 52 comprises eight grooves 68. The grooves 68 may be formed by known molding techniques. In a preferred embodiment, the eight grooves 68 are equally spaced apart from one another.

The centering pin 48 comprises an open tube 70, a lower stem section 74, and an annular seat 76, which resembles a ring formed around the stem section 74 and has a plurality of spaced-apart channels formed therein for fluid flow, as further discussed below. The tube 70 forms a central cavity 72. When the piston and pin are positioned in the valve housing 12, the tube 70 and cavity 72 extend through the interior cavity 28, and the lower stem section 74 extends through the outlet passage 44 of the valve housing 12. In the cross-sectional view shown in FIG. 1, the lower stem section 74 of the centering pin 48 does not extend all the way out to contact the lower ring section 54 of the piston 46. In this cross-section, the shape of the stem section 74 leaves a gap or flow channel 94 between the stem section 74 and the ring section 54. The stem section 74 also does not extend all the way out to the annular seat 76, leaving a lower flow channel 84. In another cross-section, taken along another plane, the lower stem section 74 of the centering pin 48 extends all the way out to the lower ring section 54 (i.e., is solid) and the gap 94 is eliminated. The stem section 74 also extends out to join with the annular seat 76, eliminating the lower flow channel 84. Thus, the flow channel 84 is not open all the way around the circumference of the valve. The structure resembles a 4 to 8 teeth spur gear viewed along a cross-section. In exemplary embodiments of the invention, a plurality of lower flow channels 84 are provided. In the particular embodiment shown in FIG. 1, there are eight lower flow channels 84 around the circumference of the centering pin 48. In a preferred embodiment, the eight lower flow channels 84 are equally spaced apart from one another.

The centering pin 48 also includes inner flow channels 82 formed in the outer surface of a top portion of the tube 70. These flow channels may be formed by forming grooves or channels or other suitable indentations in the outer surface of the tube 70. In exemplary embodiments of the invention, a plurality of inner flow channels 82 are provided in the tube 70. In the particular embodiment shown in FIG. 1, the tube 70 comprises eight inner flow channels 82. In a preferred embodiment, the eight inner flow channels 82 are equally spaced apart from one another.

In one embodiment, the piston 46 and centering pin 48 are assembled together first before being inserted into the valve housing 12. The piston 46 is mounted on the centering pin 48 such that the stem 56 enters the central cavity 72, and the tube 70 enters the annular channel 58. The middle bladder section 52 of the piston 46 encircles at least a portion of the tube 70, and the lower ring section 54 encircles at least a portion of the lower stem section 74. The lower ring section 54 sits on the annular seat 76. The piston 46 may also be secured to the pin by welding or gluing the lower ring section 54 to the lower stem section 74 along those cross sections where the stem section 74 extends all the way out to contact the ring section 54 (described above). The ring section 54 may also be welded or glued to the top surface of the annular seat 76. However, the welding or gluing is optional, and in other embodiments, the piston is not adhesively attached to the pin.

The piston 46 and centering pin 48 may be inserted together into the valve housing 12 through the inlet opening 16 until the annular seat 76 of the centering pin 48 contacts the annular step 88 of the housing 12. In this position, the lower ring section 54 of the piston 46 is wedged between the lower stem section 74 of the centering pin 48 and the lower annular shoulder 36 of the housing 12. Additionally, due to the relative dimensions of the piston 46 and the housing 12, the circumferential exterior wall surface 62 of the upper plug section 50 is urged against the interior wall 32 of the housing 12. The resiliency of the piston 46 provides a fluid tight contact between the upper plug section 50 and the interior wall 32 and is configured to terminate fluid communication between the inlet port 14 and the outlet port 18.

Thus, in the embodiment shown in FIG. 1, the piston 46 is retained inside the housing 12 by a press fit between the centering pin 48 and the housing 12 at points A and B. To further improve the effectiveness of the press fit, bumps 86 and 92 may be formed in the interior surface of the housing 12 to apply additional compression to the piston 46. These bumps may be any type of raised projection on the interior surface of the housing 12. Alternatively, in other embodiments, these bumps or projections may be absent.

The embodiment in FIG. 1 shows the piston 46 and centering pin 48 fully inserted into the valve housing 12, with the piston 46 securely wedged between the centering pin 48 and housing 12 at points A and B. In FIG. 1, the valve 10 is in a closed position. No fluid pathways are open between the inlet port 14 and the outlet port 18.

Referring now to FIG. 2, a perspective view of the valve 10 of FIG. 1 is shown. The tapered top surface 64 of the piston 46 is visible through the inlet opening 16. The step 90 and luer threads 30 are visible on the exterior surface of the valve housing 12. Although not shown, labels, aesthetic indicia, and/or ribs or projections for gripping may be incorporated on the exterior surface of the valve housing 12.

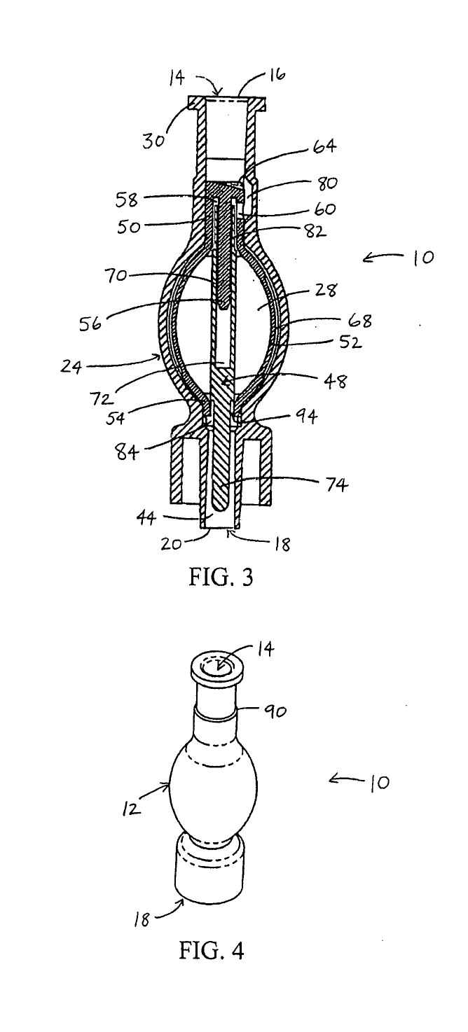

FIG. 3 is a cross-sectional side view of the valve 10 of FIG. 1 in an open position. A medical implement (not shown) is introduced at the inlet port 14 to compress the piston 46 into the open position. When the medical implement compresses the piston 46, the bottom tip of the medical implement urges against the tapered top surface 64 of the piston 46. The tapering of the surface 64 provides a flow space or gap for fluid from the medical implement to flow out of the medical implement and into the valve 10. Without tapering, the contact between the top surface 64 of the piston 46 and the medical implement could block fluid flow from the implement into the valve 10. Although tapering is described in this particular embodiment, other methods of creating a gap between the piston 46 and the medical implement can be used, such as forming grooves or channels in the top surface 64.

When the medical implement compresses the piston 46, the upper plug section 50 slides along the centering pin 48. The stem 56 slides further into the central cavity 72 of the centering pin, and the tube 70 slides up the annular channel 58. The stem 56 and central cavity 72 help guide the movement of the piston as it is compressed. When the piston is compressed, the tapered top surface 64 and the side opening 60 align with the upper flow channel 80, as shown in FIG. 3. This alignment opens a fluid pathway from the medical implement, through the gap between the medical implement and the tapered top surface 64, through the upper flow channel 80, through the side hole 60, and into the inner flow channels 82 formed in the pin 48. In the embodiment shown in FIGS. 1 and 3, only one flow channel 80 and one side opening 60 are used. However, in other embodiments, two or more side openings may be formed in the upper plug section 50 of the piston, and these side openings may align with two or more flow channels 80.

The pressure applied to the top surface 64 of the piston 46 by the medical implement causes the middle bladder section 52 of the piston to bow outward away from the centering pin 48 and toward the bulbous midsection 24 of the housing 12. The grooves 68 formed in the middle bladder section 52 of the piston 46 reduce the thickness of the piston, such that the bladder section 52 is thinner where the grooves are formed. These thinner portions of the bladder permit the middle bladder section to bulge or bend outwardly more readily when compressed by the medical implement. Thus, when the medical implement applies pressure to the top surface 64 of the piston 46, and the piston slides down the pin, the thin portions of the middle bladder section 52 bend or bulge outwardly away from the centering pin 48 toward the bulbous midsection 24 of the housing 12. The interior cavity 28 is then open between the centering pin 48 and the middle bladder section 52, as shown in FIG. 3, instead of between the bladder section 52 and the bulbous midsection 24, as shown in FIG. 1.

During this compression, the top portion of the bladder section 52 bends away from the centering pin, exposing the inner flow channels 82. The bending of the piston opens a fluid pathway between the inner flow channels 82 and the interior cavity 28. In addition, in other embodiments, grooves or channels may be formed in the interior surface of the bladder section 52 to facilitate fluid flow through the inner flow channels 82 into the interior cavity 28.

The lower ring section 54 of the piston also bends away from the centering pin when the piston is compressed by the medical implement. This bending opens a fluid pathway between the interior cavity 28 and the gap 94. Fluid can flow from the interior cavity 28 through the gap 94, into the lower flow channels 84, into the outlet passage 44, and into the IV tube (not shown). In addition, in other embodiments, grooves or channels may be formed in the interior surface of the ring section 54 to facilitate fluid flow from the interior cavity 28 through the gap 94 and into the lower flow channels 84.

When the medical implement is removed from the valve 10, the resilient piston 46 returns to the closed position shown in FIG. 1. The upper plug section 50 slides upwards along the pin 48, until the top surface 64 is once again flush with the inlet opening 16. The movement of the plug section back to this position cuts off the fluid pathway through the upper flow channel 80 and side opening 60, as the side opening 60 is no longer aligned with the upper flow channel 80. The contact between the circumferential exterior wall surface 62 of the piston and the upper interior wall 32 of the housing creates a fluid-tight seal and terminates any fluid flow between the inlet opening 16 and the outlet opening 20.

When the piston returns to the closed position, the bladder section 52 moves back across the interior cavity 28 and into contact with the tube 70 of the centering pin 48. The fluid pathway between the inner flow channels 82 and the interior cavity 28 is closed, as the upper portion of the bladder section 52 is no longer bent outward away from the flow channels 82. The fluid pathway between the interior cavity 28 and the gap 94 is also closed, as the ring section 54 of the piston is no longer bent outward away from the centering pin. Thus, when the piston returns to its closed position, the piston blocks all fluid pathways between the inlet port 14 and the outlet port 18.

When the medical implement compresses the piston 46, as shown in FIG. 3, the volume of the fluid flow space increases, because the bladder section 52 expands across the interior cavity 28 and opens the interior cavity 28 to fluid flow. This creates a negative displacement and tends to sucks a small amount of fluid into the valve from the outlet 20. When the medical implement is removed, the bladder section 52 of the piston 46 moves back toward the centering pin 48, thereby decreasing the volume of the fluid flow space. This causes the valve 10 to flush out the fluid inside the interior cavity 28. The fluid is forced through the gap 94, into the lower flow channels 84, and out the outlet passage 44. The valve 10 thus operates as a self-flushing or positive displacement valve. The lower stem section 74 of the centering pin 48 helps to flush fluid from the valve 10 by occupying volume in the outlet passage 44. The lower stem section 74 thus displaces fluid that may otherwise occupy the outlet passage 44.

Referring now to FIG. 4, a perspective view of the valve 10 of FIG. 3 is shown. The top tapered surface 64 of the piston 46 is not visible through the inlet port 14 because it has been compressed by the medical implement (not shown).

Although limited embodiments of the needleless access port valve and its components have been specifically described and illustrated herein, many modifications and variations will be apparent to those skilled in the art. For example, the valve housing may be made from two or more housing components, the inlet can be a luer slip rather luer lock, and fewer or more flow channels may be incorporated than as described. Accordingly, it is to be understood that the valve and its components constructed according to principles of this invention may be embodied other than as specifically described herein. The invention is also defined in the following claims.

What is claimed is:

1. A needleless access port valve comprising:
a valve housing comprising an inlet port adapted to receive a medical implement, an outlet port, and an interior wall surface defining an interior cavity;
a resilient piston having a generally tubular shape wall structure defining an interior space, wherein the resilient piston comprises a neck section, a middle section, and a lower section;
a pin occupying, at least in part, the interior space of the resilient piston;
wherein the lower section of the resilient piston is compressed between the pin and the housing; and
wherein the neck section of the resilient piston forms a seal with the housing to prevent fluid flow between the inlet port and the outlet port when in a closed position.

2. The needleless access port valve of claim 1, wherein the resilient piston comprises a side opening spaced from an upper end surface.

3. The needleless access port valve of claim 1, wherein the pin comprises a lower stem section extending into the outlet port.

4. The needleless access port valve of claim 1, wherein the resilient piston comprise a stem located inside the tubular shape wall structure.

5. The needleless access port valve of claim 1, further comprising at least one upper flow channel formed in the interior wall surface of the valve housing.

6. The needleless access port valve of claim 1, wherein the inlet port comprises a Luer taper.

7. The needleless access port valve of claim 1, wherein the middle section of the resilient piston expands outwardly when an upper surface of the resilient piston is compressed by a medical implement.

8. A needleless access port valve comprising
a valve housing having an inlet port, an outlet port, an interior cavity, and at least one flow channel;
a pin;
a resilient piston formed over the pin and positioned in the interior cavity of the housing;
wherein the piston comprises an upper neck section, a mid-section, and a lower section;
wherein the upper neck section comprises an opening configured to align with the flow channel of the valve housing when the valve is in an open position;
wherein the lower section is compressed between the pin and the housing to hold the piston in place; and
wherein the mid-section is configured to expand outwardly away from the pin when the piston is compressed to open a fluid pathway between the inlet port and the outlet port.

9. The needleless access port valve of claim 8, wherein the pin comprises a central cavity.

10. The needleless access port valve of claim 8, wherein the pin comprises a lower stem section extending into the outlet port.

11. The needleless access port valve of claim 8, wherein the resilient piston comprises a tubular shape wall structure defining an interior space having a stem located therein.

12. The needleless access port valve of claim 8, the at least one flow channel is formed into an interior wall surface of the valve housing.

13. The needleless access port valve of claim 8, wherein the inlet port comprises a Luer taper.

14. A needleless access port valve comprising:
a valve housing comprising an inlet port, an outlet port, an upper neck section, and an interior cavity, wherein the upper neck section comprises at least one flow channel;
a pin; and
a resilient piston slidably mounted on the pin and positioned in the interior cavity of the valve housing, wherein the piston comprises an upper sliding section, a middle expanding section, and a lower mounting section;
wherein the lower mounting section is compressed between the pin and the housing, and wherein the middle expanding section is adapted to expand away from the pin when the piston is compressed, and wherein the upper sliding section is configured to slide along the pin when the piston is compressed; and
wherein the upper sliding section of the piston comprises a side opening, the side opening configured to align with the flow channel when the piston is compressed to open a fluid pathway between the inlet port and the outlet port.

15. The needleless access port valve of claim 14, wherein the middle expanding section has a wall thickness that is less than a wall thickness of the upper neck section.

16. The needleless access port valve of claim 14, wherein the pin comprises a lower stem section extending into the outlet port.

17. The needleless access port valve of claim 14, wherein the resilient piston comprises a tubular shape wall structure defining an interior space having a stem located therein.

18. The needleless access port valve of claim 14, wherein the valve housing comprises an integrally formed threaded collar.

19. The needleless access port valve of claim 14, wherein the inlet port comprises a Luer taper.

20. The needleless access port valve of claim 14, further comprising a second side opening.
